(19) United States
(12) Patent Application Publication
(10) Pub. No.: US 2022/0233459 A1
(43) Pub. Date: Jul. 28, 2022

(54) ANALYSIS OF MATERIALS FOR TISSUE DELIVERY

(71) Applicant: Guide Therapeutics, Inc., Cambridge, MA (US)

(72) Inventors: Cory Dane Sago, Atlanta, GA (US); Milloni Balwantkumar Chhabra, Dunwoody, GA (US)

(21) Appl. No.: 17/596,187

(22) PCT Filed: Jun. 2, 2020
(86) PCT No.: PCT/US2020/035730

§ 371 Date: Dec. 3, 2021

Related U.S. Application Data
(60) Provisional application No. 62/857,676, filed on Jun. 5, 2019.

(51) Int. Cl. A61K 9/51; C12Q 1/6816; A61K 47/34; A61K 9/00; C12N 15/88; A61K 31/7105

(52) U.S. Cl. CPC A61K 9/5123; C12Q 1/6816; A61K 47/34; C12N 2320/32; C12N 15/88; A61K 31/7105; A61K 9/0019

(57) ABSTRACT

Described herein are compositions and methods for identifying materials suitable for delivery of an agent to a target tissue. These compositions and methods may simultaneously screen a library of materials for the ability to deliver an agent to a target. The compositions and methods may also be used to confirm that the agent is delivered in a manner sufficient for function of the agent.

### FIELD

The present disclosure is directed to methods and compositions for characterizing delivery vehicles, including but not limited to lipid nanoparticle delivery vehicles.

### BACKGROUND

The development of nanoparticles for the treatment and detection of human diseases is expected to result in an explosion of the market for this class of biomaterials. Nanoparticles carrying mRNA encounter dynamic hurdles evolved to prevent foreign nucleic acid delivery. To overcome these challenges, Lipid Nanoparticles (LNPs) are imparted with chemical diversity two ways. First, thousands of compounds with variable ionizability, pKa, and hydrophobicity can be synthesized. Second, each compound can be formulated into hundreds of chemically distinct LNPs by adding poly(ethylene glycol) (PEG), cholesterol, 1,2-dioleoyl-sn-glycero-3-phosphoethanolamine (DOPE), or other constituents.

Nanoparticle libraries, comprising hundreds to thousands of LNPs, can be screened in vitro. This process is more effective if it predicts in vivo (in a living animal) delivery. In vivo mRNA delivery may be affected by pulsatile blood flow, heterogenous vasculature, and clearance by the kidney, spleen, liver, lymphatics, and immune system. Barcoding technologies have quantified LNP biodistribution, which is necessary, but not sufficient, for cytoplasmic nucleic acid delivery. More specifically, less than 3% of a drug that reaches a target cell generally escapes into the cytoplasm, and the genes that alter whether the nanoparticle escapes into the endosome are likely to vary with each cell type. As a result, it is difficult to predict functional delivery of drug into the cytoplasm or nucleus by measuring biodistribution alone.

To overcome these obstacles, there is a need for a method for characterizing and screening delivery vehicles that exhibit a desired tropism and deliver functional cargo to a specific cell or tissue.

### SUMMARY

The claims below describe improvements to the subject matter of published PCT Application WO 2019/089561 (hereafter, “the '561 Application”), which is incorporated herein by reference. Various terms used in the claims have their ordinary meaning as understood by persons of ordinary skill in the art and thus include the definitions set forth in the '561 Application. For example, CD47 and CD81 are cluster of differentiation proteins that are present on the surface of various cells in the bodies of mammalian subjects.

Methods for characterizing particle delivery vehicles are described in the claims below, and additional embodiments are described herein. The described methods use biologically active molecules that have functionality that can be detected when the molecules are delivered to a particular cell or tissue type. Detecting the function of the biologically active molecules in the cell indicates that the formulation of a corresponding delivery vehicle is capable of delivering functional cargo to the cell. Representative biologically active molecules that may be used in these methods include, but are not limited to: siRNA, an antisense oligonucleotide, mRNA, DNA transgene, nuclease protein, nuclease mRNA, small molecules, epigenetic modifiers, and phenotypic modifiers.

In various embodiments, the biologically active molecule is selected on the basis that it generates a detectable signal when delivered by the LNP delivery vehicle to the cytoplasm of cells of at least two species of non-human mammals. Thus, a LNP delivery vehicle that comprises such a biologically active molecule and is found to be capable of delivery to a particular cell type or tissue in a first species of non-human mammal (e.g., mouse or rat) will also be capable of delivery to the corresponding cell type or tissue in a second species of non-human mammal (e.g., a non-human primate).

In one embodiment, the administration of the biologically active molecule results in down regulation that results in reduced expression of beta-2-microglobulin.

In another embodiment, the administration of the biologically active molecule results in down regulation that results in reduced expression of CD47.

In another embodiment, the administration of the biologically active molecule results in down regulation that results in reduced expression of CD81.

In another embodiment, the administration of the biologically active molecule results in down regulation that results in reduced expression of AP2S1.

In another embodiment, the administration of the biologically active molecule results in down regulation that results in reduced expression of LGALS9.

In another embodiment, the administration of the biologically active molecule results in down regulation that results in reduced expression of ITGB1.

In another embodiment, the administration of the biologically active molecule results in down regulation that results in reduced expression of ITGA5.

In another embodiment, the administration of the biologically active molecule results in down regulation that results in reduced expression of CD45.

In another embodiment, the administration of the biologically active molecule results in down regulation that results in reduced expression of TIE2.

In another embodiment, the administration of the biologically active molecule results in down regulation that results in reduced expression of MGAT4B.

In another embodiment, the administration of the biologically active molecule results in down regulation that results in reduced expression of MGAT2.

In another embodiment, the administration of the biologically active molecule results in down regulation that results in reduced expression of VAMP3.

In another embodiment, the administration of the biologically active molecule results in down regulation that results in reduced expression of GPAA1.

In some embodiments, a chemical composition identifier may be included in each different delivery vehicle formulation to identify the chemical composition specific to each different delivery vehicle formulation. For example, the chemical composition identifier may be a nucleic acid barcode. The sequence of the nucleic acid barcode is correlated with the chemical components used to formulate the delivery vehicle in which it is loaded so that when the nucleic acid barcode is sequenced, the chemical composition of the delivery vehicle that delivered the barcode is identified.

One embodiment of an LNP for delivery of siRNA or an antisense oligonucleotide (ASO) as a biologically active molecule is an LNP that comprises a two-component system in which the barcode is separate from the siRNA or ASO, as exemplified by the following:
siRNA+Barcode

One embodiment of an LNP for delivery of mRNA as a biologically active molecule is an LNP that comprises a two-component system, as exemplified in FIG. 5.

In various embodiments, the barcode can be incorporated into the biologically active molecule or the barcode can be separate from the biologically active molecule, as exemplified in various embodiments illustrated in FIG. 6.

Compositions and methods for characterizing delivery vehicles that deliver functional cargo are provided. Many delivery vehicles are able to deliver cargo to cells, but the cargo may be trapped in an endosome or lysosome and is effectively rendered non-functional. The disclosed compositions and methods advantageously have the ability to assay multiple delivery vehicle formulations in a single run that not only deliver the agent to a desired cell or tissue, but are also able to identify delivery vehicle formulations that deliver cargo in its functional form. For example, if the cargo is a nucleic acid, expression of the nucleic acid in the cell shows that the nucleic acid is functional when delivered to the cytoplasm or nucleus of the cell.

In one embodiment, the method includes a delivery vehicle that contains a reporter and a chemical composition identifier. The method includes the step of formulating multiple delivery vehicles having different chemical compositions. In one embodiment >100 or even greater than >250 different delivery vehicle formulations are assayed in one run. The delivery vehicles are formulated to be taken up by cells. The delivery vehicles contain a reporter that can generate a detectable signal when it is functionally delivered into the cytoplasm or nucleus of cells of a non-human animal, and a composition identifier that identifies the chemical composition of the delivery vehicle. The reporter can be a nucleic acid such as mRNA that encodes a protein that when expressed in a cell is able to generate a detectable signal. For example, the protein can be a fluorescent protein or an enzyme the produces a detectable substance in the cell.

The method also includes the steps of pooling and administering the multiple delivery vehicles to a non-human mammal, for example a laboratory animal such as a mouse, rat, or non-human primate. After administration of the multiple delivery vehicles, cells from multiple tissues of the non-human mammal that generate the detectable signal are sorted from cells that do not generate the detectable signal. In one embodiment, the cells are sorted using fluorescence activated cell sorting (FACS). In some embodiments, the cells that generate the detectable signal are also sorted based on the presence or absence of a cell surface protein that is indicative of tissue type or cell type. Representative cell surface proteins include, but are not limited to, cluster of differentiation proteins. Fluorophore-conjugated antibodies to the cell surface proteins are used to detect the cell surface proteins on the cells and sort the cells.

The method also includes the step of identifying the chemical composition identifier in the sorted cells that generate the detectable signal to determine the chemical composition of the delivery vehicles in the sorted cells and to correlate the chemical composition of the delivery vehicles to the tissue or cell type containing the particles based on the cell surface markers on the sorted cells. In one embodiment the chemical composition identifier is a nucleic acid barcode, and the sequence is determined for example using deep sequencing techniques (also referred to as high-throughput sequencing or next generation sequencing).

Once the delivery vehicles are characterized, they can be used to deliver cargo to the cells of a subject in need thereof. The cargo can be a biologically active agent including, but not limited to nucleic acids and proteins. Exemplary agents include, but are not limited to mRNA, siRNA, nucleases, recombinases, and combinations thereof.

In some embodiments, the delivery vehicles are particles, for example nanoparticles. Nanoparticles typically have a diameter of less than 1 micron. In one embodiment, the nanoparticles have a diameter of 20 nm to 200 nm. In one embodiment, the particles are lipid nanoparticles.

In some embodiments, the delivery vehicle is a conjugate containing three components: (1) a reporter; (2) a chemical composition identifier; and (3) one of the group consisting of a peptide, a lipid, ssRNA, dsRNA, ssDNA, dsDNA, or a polymer. The three components can be in any arrangement in the conjugate. Exemplary reporters include, but are not limited to siRNA, mRNA, nuclease mRNA, small molecules, epigenetic modifiers, and phenotypic modifiers. An epigenetic modifier is a molecule that can cause a detectable change in the structure of DNA inside the cell when the molecule is delivered to the cell. An exemplary epigenetic modifier includes a protein that alters the chromatin structure of DNA inside a cell in a way that can be analyzed using DNA sequencing (e.g., ATAC-seq). A phenotypic modifier is a molecule that can cause a detectable change in the structure or behavior of a cell when the molecule is delivered to the cell. An exemplary phenotypic modifier includes a molecule that induces a change in the cell, for example cell morphology. The chemical composition identifier can be a nucleic acid barcode as discuss above.

Another embodiment provides a composition containing a delivery vehicle, a nucleic acid bar code, and a reporter that is biologically active when delivered to the cytoplasm or nucleus of a cell. In some embodiments, the delivery vehicle is a lipid nanoparticle. In other embodiments, the delivery vehicle is a conjugate.

Still another embodiment provides a nucleic acid barcode composition according to the following formula

R1-R2-R3-R4-R5-R6-R7-R8-R1

wherein

R1 represents 1, 2, 3, 4, 5, 6, 7, 8, 9, or 10 nucleotides with phosphorothioate linkages,

R2 represents a first universal primer binding site,

R3 represents a spacer,

R4 represents a digital droplet PCR probe binding site,

R5 represents a random nucleotide sequence;

R6 represents a nucleic acid barcode sequence,

R7 represents a random nucleic acid sequence; and

R8 represents a second universal primer binding site.

Another embodiment provides a pharmaceutical composition containing one or more of the nucleic acid barcodes disclosed herein.

The details of one or more embodiments are set forth in the accompanying drawings and the description below. Other features, objects, and advantages of the embodiments will be apparent from the description and drawings, and from the claims.

### BRIEF DESCRIPTION OF THE DRAWINGS

FIG. 1 is a plot showing the diameter distribution of 192 LNPs formulated to carry siCD45 and DNA barcodes at a mass ratio of 10:1.

FIG. 2 is a histogram showing the concentration of encapsulated and unencapsulated siRNA in the pool of 192 LNPs.

FIG. 3 is a plot showing the normalized fold above input relating LNP delivery between bone marrow tissue extracted from two distinct rats that had been administered a pool of 201 LNPs carrying siRNA and CD45 at a dose of 1.5 mg/kg siRNA.

FIG. 4 is a plot showing the normalized fold above input relating LNP delivery between FACS isolated bone marrow monocytes extracted from two distinct non-human primates (NHPs) that had been administered a pool of 201 LNPs carrying siRNA and CD45 at a dose of 1.5 mg/kg siRNA.

FIG. 5 is a diagram depicting a two-component system for LNP delivery of mRNA.

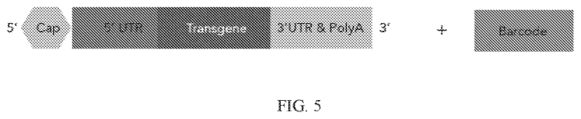

FIG. 6 is a diagram depicting various options for incorporating a barcode into a biologically active molecule or keeping it separate.

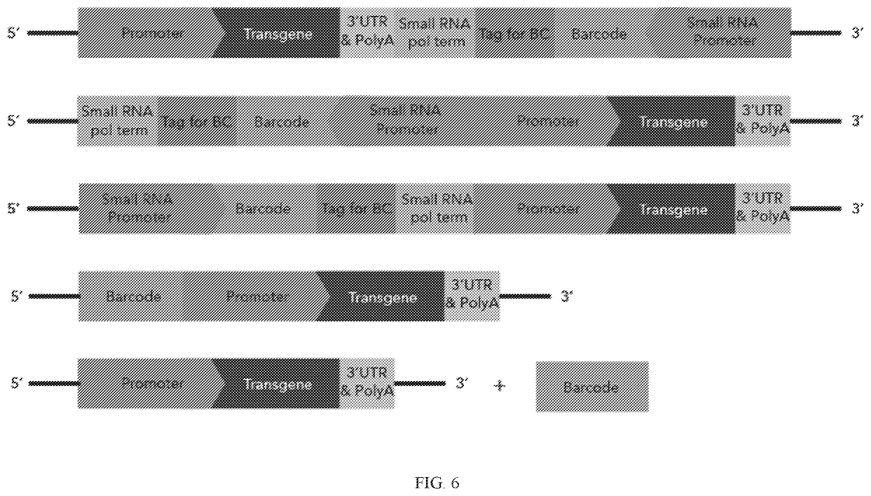

### DETAILED DESCRIPTION

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It must be noted that, as used in the specification and the appended claims, the singular forms “a,” “an,” and “the” include plural referents unless the context clearly dictates otherwise.

### I. Definitions

As used herein, “bioactive agent” is used to refer to compounds or entities that alter, inhibit, activate, or otherwise affect biological or chemical events. For example, bioactive agents may be chemical entities or biological products that have therapeutic or diagnostic activity when delivered to a cell in a subject. The chemical entity or biological product can be an organic or inorganic molecule. In some embodiments, the bioactive agent is a modified or unmodified polynucleotide. In some embodiments, the bioactive agent is a peptide or peptidomimetics. In some cases, the bioactive agent is a protein. In some embodiments, the bioactive agent is an antisense nucleic acid, RNAi (e.g. siRNA, miRNA or shRNA), receptor, ligand, antibody, aptamer, or a fragment, analogue, or variant thereof. In some embodiments, the bioactive agent is a vector comprising a nucleic acid encoding a therapeutic or diagnostic gene. Bioactive agents may include, but are not limited to, anti-AIDS substances, anti-cancer substances, antibiotics, immunosuppressants, anti-viral substances, enzyme inhibitors, including but not limited to protease and reverse transcriptase inhibitors, fusion inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents. In a certain embodiments, the bioactive agent is a drug. A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, and the United States Pharmacopeia-25/National Formulary-20, published by the United States Pharmcopeial Convention, Inc., Rockville Md., 2001, all of which are incorporated herein by reference.

The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant techniques) that are commonly found in nature (e.g., organisms, tissues, cells, or viruses). Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, siRNA, mRNA, miRNA, DNA, and RNA.

As used herein, “biodegradable” polymers are polymers that degrade (i.e., down to monomeric species or oligomers that can be eliminated or processed by the body) under physiological conditions. In some embodiments, the polymers and polymer biodegradation byproducts are biocompatible. Biodegradable polymers are not necessarily hydrolytically degradable and may require enzymatic action to fully degrade. In certain embodiments, the biodegradable polymer is degraded by the endosome

As used herein, the term “functionally expressed” refers to a coding sequence which is transcribed, translated, post-translationally modified (if relevant), and positioned in a cell such that the protein functions.

The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least two nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, 2′-methoxyribose, 2′-aminoribose, ribose, 2′-deoxyribose, arabinose, and hexose), unnatural base pairs (UBPs), or modified phosphate groups (e.g., phosphorothioates and 5′-N phosphoramidite linkages). Enantiomers of natural or modified nucleosides may also be used. Nucleic acids also include nucleic acid-based therapeutic agents, for example, nucleic acid ligands, siRNA, short hairpin RNA, antisense oligonucleotides, ribozymes, aptamers, and SPIEGELMERS™, oligonucleotide ligands described in Wlotzka, et al., Proc. Natl. Acad. Sci. USA, 2002, 99(13):8898, the entire contents of which are incorporated herein by reference. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

The terms “polypeptide”, “peptide”, and “protein”, may be used interchangeably to refer a string of at least three amino acids linked together by peptide bonds. Peptide may refer to an individual peptide or a collection of peptides. Peptides can contain natural amino acids, non-natural amino acids (i.e., compounds that do not occur in nature but that can be incorporated into a polypeptide chain), and/or amino acid analogs. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. Modifications may include cyclization of the peptide, the incorporation of D-amino acids, etc.

As used herein, “peptidomimetic” refers to a mimetic of a peptide which includes some alteration of the normal peptide chemistry. Peptidomimetics typically enhance some property of the original peptide, such as increase stability, increased efficacy, enhanced delivery, increased half-life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-a-amino butyric acid, L-v-amino butyric acid, L-a-amino isobutyric acid, L-amino caproic acid, 7-amino heptanoic acid, L-aspartic acid, L-glutamic acid, N-ε-Boc-N-α-CBZ-L-lysine, N-ε-Boc-N-a-Fmoc-L-lysine, L-methionine sulfone, L-norleucine, L-norvaline, N-α-Boc-N-5CBZ-L-ornithine, N—δ-Boc-N-α-CBZ-L-ornithine, Boc-p-nitro-L-phenylalanine, Boc-hydroxyproline, and Boc-L-thioproline.

The terms “polysaccharide”, “carbohydrate”, or “oligosaccharide” may be used interchangeably to refer to a polymer of sugars. Typically, a polysaccharide comprises at least two sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

As used herein, the term “small molecule” is used to refer to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, heterocyclic rings, etc.). In some embodiments, small molecules are monomeric and have a molecular weight of less than about 1500 g/mol. In certain embodiments, the molecular weight of the small molecule is less than about 1000 g/mol or less than about 500 g/mol. Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “subject” refers to any individual who is the target of administration or treatment. The subject can be a vertebrate, for example, a mammal and particularly a human. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

### II. Methods for Characterizing Particle Delivery Vehicles

Methods and compositions for characterizing vehicle delivery formulations to identify formulations with a desired tropism and that deliver functional cargo to the cytoplasm of specific cells are provided. The disclosed methods and compositions use a reporter that has a functionality that can be detected when delivered to the cell. Detecting the function of the reporter in the cell indicates that the formulation of the delivery vehicle will deliver functional cargo to the cell. A chemical composition identifier is included in each different delivery vehicle formulation to keep track of the chemical composition specific for each different delivery vehicle formulation. In one embodiment, the chemical composition identifier is a nucleic acid barcode. The sequence of the nucleic acid bar code is paired to the chemical components used to formulate the delivery vehicle in which it is loaded so that when the nucleic acid bar code is sequenced, the chemical composition of the delivery vehicle that delivered the barcode is identified. Representative reporters include, but are not limited to siRNA, mRNA, nuclease protein, nuclease mRNA, small molecules, epigenetic modifiers, and phenotypic modifiers.

A. In vivo Methods

One embodiment provides an in vivo method for characterizing delivery vehicle formulations for in vivo delivery of an agent including the steps of formulating multiple delivery vehicles having different chemical compositions, wherein each delivery vehicle contains a reporter that can generate a detectable signal when delivered to the cytoplasm of cells of a non-human mammal, and a composition identifier that identifies the chemical composition of the vehicle. The method also includes the steps of pooling and administering the multiple delivery vehicles to a non-human mammal. The method also includes the step of sorting cells from multiple tissues of the non-human mammal that generate the detectable signal from cells that do not generate the detectable signal, wherein the cells that generate the detectable signal are also sorted based on the presence or absence of a cell surface protein that is indicative of tissue type or cell type. After the cells are sorted, the method includes the step of identifying the chemical composition identifier in the sorted cells that generate the detectable signal to determine the chemical composition of the delivery vehicle in the sorted cells and correlate the chemical composition of the delivery vehicle with the tissue or cell type containing the delivery vehicle. In some embodiments, the delivery vehicle is a particulate delivery vehicle, and in other embodiments the delivery vehicle is a conjugate. In some embodiments, the method is a high-throughput screening assay.

The pool of multiple delivery vehicle formulations is typically administered parenterally, for example by intravenous injection or intramuscular injection.

Alternatively, the composition may be administered by other routes, e.g., intraarterial, inhalational, intradermal, subcutaneous, oral, nasal, bronchial, ophthalmic, transdermal (topical), transmucosal, peritoneal, rectal, and vaginal routes. In some embodiments, the materials are not only optimized to reach a particular tissue site but for a particular delivery route.

After a defined period of time post-administration, the tissues or cells are harvested and processed for sorting. In some cases, targeted cells positive for the reporter or label are isolated. In other cases, targeted cells negative for the reporter or label are isolated, e.g., wherein the materials contain an inhibitor of a constitutive reporter transgene. The materials that are present in those cells can then be isolated for identification. In some embodiments, the materials are processed to release the associated barcodes, which are used to identify the materials that were present in the tissue. The amount of total materials present per cell may also be quantified. Alternatively or in addition, samples from non-targeted cells or organs can be collected, and the materials identified by the same process. This way, those materials with undesirable biophysiochemical properties, such as non-specific tissue targeting, may be identified and eliminated from subsequent rounds of enrichment.

In some embodiments, target cells are assayed to identify the nucleic acid barcodes present in the cells, thereby identifying the corresponding materials. In some cases, this involves sequencing the barcodes, e.g. using PCR amplification, followed by next generation sequencing (NGS or deep sequencing).

The protocols used for reporter positive cell isolation will vary based on the reporter system used, as well the cell source (e.g. in vivo tissue/blood and in vitro cell culture). Tissues and cells may be isolated with the animal alive or post-mortem. Whole or partial tissue and organs may be extracted from the animal. Biopsies may be the source of cells. Cells may be isolated from blood from various routes including cardiac puncture or retro orbital blood draw. Isolation may occur via enzymatic (e.g. trypsin, various collagenases, and combinations) and/or mechanical methods (e.g., centrifugation, mortar and pestle, chopping, and grinding). The resulting cell suspensions may be either heterogeneous or homogenous cell types depending on source. These suspensions can then be separated based on a multitude of criteria (e.g., cell type, cell markers, cell cycle, reporter status) simultaneously or in sequential manner. This may be done by fluorescent assisted cell sorting, magnetic assisted cell sorting, centrifugation, and affinity based cell isolation (e.g., antibody-DNA conjugates, antibody-biotin). Cells can be isolated into single-cell or bulk populations. Barcodes are then isolated from the cell. This can be done via chromatography or solution-based methods. Barcodes may be first separated from genomic DNA via size differences or other characteristics, or genomic DNA can be degraded; alternatively, genomic DNA may be left unperturbed. Extracted barcodes can be left concentrated or diluted for further analysis. This barcode extract can be sequenced directly or amplified by PCR to make more copies. Barcodes can be sequenced by Sanger sequencing, Next-Generation Sequencing (e.g., Illumina, Roche 454, Ion torrent), or Nanopore-based sequencing methods.

Those formulations that demonstrate functional targeting of the desired tissue, while optionally demonstrating a low level of uptake by non-targeted organs may be enriched. The screening may be repeated several times, for example, to improve the resolution of the assay. In addition, the strength of the screen may be modified by requiring higher or lower levels of signal from a particular label in order to select the corresponding material for enrichment.

In some embodiments, the method further involves creating or producing a new library of delivery vehicles based on those shown to demonstrate functional targeting. The disclosed method in this way can be used to optimize the biophysical characteristics of the materials. Parameters for optimization may include but are not limited to any of size, polymer composition, surface hydrophilicity, surface charge, and the presence, composition and density of targeting agents on the material surface. The new library can be assayed as above and used to determine which optimizations were effective.

In one embodiment, the delivery vehicles are nanoparticles formulated using a microfluidic device. Nanoparticle 1, with chemical composition 1, is formulated to carry reporter mRNA and barcode 1. Nanoparticle 2, with chemical composition 2, is formulated to carry reporter mRNA and barcode 2. This process is repeated N times, such that Nanoparticle N, with chemical composition N, is formulated to carry reporter mRNA and barcode N. The chemical components making up nanoparticle 1 are loaded into one glass syringe. The barcode 1 and reporter mRNA are loaded into a separate syringe. The contents of the syringes are mixed together at flow rates of 200 μL/min for the nanoparticle syringe and 600 μL/min for the barcode and reporter mRNA syringe. Nanoparticles are then characterized by diluting them into sterile 1×PBS at a concentration of 0.00001 to 0.01 mg/mL. At this point, the hydrodynamic diameter of the nanoparticles as well as their autocorrelation curves are analyzed using DLS. The nanoparticles are then dialyzed into a regenerated cellulose membrane, and then dialyzed into a large molecular weight (>100 kDa) cellulose membrane. The nanoparticles are then sterile filtered through a 0.22 μm filter, and loaded into a sterilized plastic tube.

The nanoparticles are then administered to mice, and a timepoint between 2 hours and 168 hours later, the mice are sacrificed.

In one embodiment, the reporter mRNA encodes GFP; in this case, GFP⁺ cells would be isolated and the timepoint would range between 2 and 48 hours.

In another embodiment, the reporter mRNA encodes tdTomato. In this case, tdTomato⁺ cells are isolated and the timepoint would range between 2 and 120 hours. In another embodiment, the reporter is RFP. RFP⁺ cells are isolated and the timepoint would range between 2 and 48 hours.

In another embodiment, the reporter is BFP. In this case, BFP⁺ cells are isolated and the timepoint would range between 2 and 48 hours.

In another embodiment, the reporter is ICAM-2, which is a gene that is expressed on the cell surface. In this case, ICAM-2⁺ cells are isolated using an ICAM-2 antibody (BioLegend clone 3C4) and the timepoint would range between 2 and 48 hours.

In another embodiment, the reporter is MHC1, which is a gene that can be expressed on the cell surface. In this case, MHC1⁺ cells are isolated from a MHC2⁺ mouse strain (i.e., 002087) using a MHC1 antibody (Clone ERMP42) and the timepoint would range between 2 and 48 hours.

In another embodiment, the reporter is MHC2, which is a gene that can be expressed on the cell surface. In this case, MHC2⁺ cells are isolated from a MHC1 mouse strain (i.e., 003584) using a MHC2 antibody (Clone IBL-5/22) and the timepoint would range between 2 and 48 hours.

In another embodiment, the reporter is Firefly Luciferase, which is a protein that is expressed in the cytoplasm. In this case, Luciferase⁺ cells are isolated using a Luciferase antibody (Clone C12 or polyclonal) and the timepoint would range between 2 and 48 hours.

In another embodiment, the reporter is Renilla Luciferase, which is a protein that is expressed in the cytoplasm. In this case, Luciferase⁺ cells are isolated using a Luciferase antibody (Clone EPR17792 or polyclonal) and the timepoint would range between 2 and 48 hours.

In yet another embodiment, the reporter is Cre. In this case, the nanoparticles are injected into a Cre reporter mouse (for example, the Lox-Stop-Lox-tdTomato Ai14 mouse strain) and tdTomato⁺ cells are isolated, and the timepoint would range between 2 and 120 hours.

In one embodiment, the reporter siRNA is siGFP. In this case, the nanoparticles are administered to a GFP-positive mouse (e.g. JAX 003291). GFP^{low} cells are isolated and the timepoint would range between 2 and 96 hours.

In another embodiment, the reporter is siRFP; in this case, the nanoparticles are administered to a RFP-positive mouse (e.g. JAX 005884). RFP^{low} cells are isolated and the timepoint would range between 2 and 96 hours.

In another embodiment, the reporter is silCAM-2, which is a gene that is expressed on the cell surface. In this case, ICAM-2^{low} cells are isolated using an ICAM-2 antibody (BioLegend clone 3C4) and the timepoint would range between 2 and 96 hours.

In another embodiment, the reporter is siCD45, which is a gene that is expressed on the cell surface. In this case, CD451 cells are isolated using a CD45 antibody (BioLegend clone 1021 and the timepoint would range between 2 and 96 hours. In another embodiment, the reporter is siCD47, which is a gene that is expressed on the cell surface. In this case, CD47^{low} cells are isolated using a CD47 antibody (BioLegend clone miap301) and the timepoint would range between 2 and 96 hours.

In another embodiment, the reporter is siTie2, which is a gene that is expressed on the cell surface. In this case, Tie2^{low} cells are isolated using a Tie2 antibody (BioLegend clone TEK4) and the timepoint would range between 2 and 96 hours. In other embodiments, the reporter siRNA is a microRNA.

In one embodiment, the reporter sgRNA is sgGFP. In this case, the nanoparticles are administered to a Cas9-GFP expressing mouse (e.g. JAX 026179). GFPol^{low} cells are isolated and the timepoint would range between 2 and 120 hours.

In another embodiment, the reporter is sglCAM-2 and is injected into Cas9 expressing mice, which is a gene that is expressed on the cell surface. In this case, ICAM-2^{low} cells are isolated using an ICAM-2 antibody (BioLegend clone 3C4) and the timepoint would range between 2 and 120 hours.

In another embodiment, the reporter is sgCD45 and is injected into Cas9 expressing mice, which is a gene that is expressed on the cell surface. In this case, iCD45^{low} cells are isolated using a CD45 antibody (BioLegend clone 102) and the timepoint would range between 2 and 120 hours.

In another embodiment, the reporter is sgCD47 and is injected into Cas9 expressing mice, which is a gene that is expressed on the cell surface. In this case, CD47^{low} cells are isolated using a CD47 antibody (BioLegend clone miap301) and the timepoint would range between 2 and 96 hours.

In another embodiment, the reporter is sgTie2 and is injected into Cas9 expressing mice, which is a gene that is expressed on the cell surface. In this case, Tie2^{low} cells are isolated using a Tie2 antibody (BioLegend clone TEK4) and the timepoint would range between 2 and 120 hours.

In another embodiment, the reporter is sgLoxP and is injected into Cas9-Lox-Stop-Lox-tdTomato expressing mice. tdTomato⁺ cells are isolated and the timepoint would range between 2 and 120 hours.

At the appropriate timepoint, the tissues from the mice are digested, and cells that are positive for the functional reporter molecule are isolated. In some embodiments, the cells are isolated by sacrificing the animal, dissecting the tissues, and adding enzymes to digest the tissues including but not limited to the following: Collagenase Type I, IV, XI, and Hyaluronidase. The tissues are then shaken at a temperature of 37° C. for 15-60 minutes, and strained through a 40, 70, or 100 μm strainer to isolate individual cell types. In some embodiments the cells are sorted by cell type or tissue type using a fluorescence activated cell sorter.

The cells are then lysed to isolate the barcodes inside. In some embodiments, cells are exposed to DNA-extraction protocols, for example QuickExtract™. In this embodiment, the cells are then prepared for DNA sequencing using PCR that adds indices that indicate the sample, purified using magnetic beads, added to PhiX control sequences (if using an Illumina machine) diluted to 4 nM concentrations, and sequenced using a MiniSeq®, MiSeq®, NextSeq®, or other next generation sequencing machine.

In other embodiments, cells are exposed to RNA-extraction protocols, for example OligoTex® kits. In this embodiment, reverse transcriptase is applied to the cells to convert any RNA to cDNA. At this point, the cDNA is prepared for sequencing using PCR that adds indices that indicate the sample, purified using magnetic beads, added to PhiX@control sequences (if using an lllumina machine) diluted to 4 nM concentrations, and sequenced using a MiniSeq®, MiSeq®, NextSeq®, or other next generation sequencing machine.

### B. In Vitro Methods

Another embodiment provides an in vitro method of characterizing the delivery vehicle formulations. In this embodiment cells or a cell line can be used that contain a gene that has been modified to prevent expression of the gene, for example a gene that encodes a fluorescent protein. The reporter in the delivery vehicle can be a recombinase or nuclease or nucleic acids that encode the recombinase or nuclease. When the delivery vehicle delivers the reporter to the cells, the recombinase or nuclease repairs the modified gene so that the fluorescent protein is expressed. The cells can be a heterogeneous pool of cells from several different tissues. After administration of the delivery vehicles the cells can be sorted to identify the cells that fluoresce and for tissue or cell type. Nucleic acid bar codes can be isolated form the different types of cells, sequenced to identify the chemical composition of the delivery vehicles that delivered them.

### III. Delivery Vehicles

### A. Representative Delivery Vehicles

Another embodiment provides a composition containing a delivery vehicle, a chemical composition identifier, for example a nucleic acid bar code, and a reporter that is biologically active when delivered to the cytoplasm of a cell. The composition optionally contains a targeting agent. In some embodiments, the delivery vehicle is a lipid nanoparticle. In other embodiments, the delivery vehicle is a conjugate. The reporter can be siRNA, mRNA, a nuclease, a recombinase, a small molecule, an epigenetic modifier, or a combination thereof.

In one embodiment, the delivery vehicle contains a pegylated C6 to C18 alkyl, cholesterol, DOPE, a chemical composition identifier and reporter. In still other embodiments, the delivery vehicle is a conjugate.

1. Nanoparticle Delivery Vehicles

The following exemplary delivery vehicles can be used in the disclosed compositions and methods and contain a reporter and a chemical composition identifier. In some embodiments, the delivery vehicle is a lipidoid nanoparticle as described in Turnbull I C, et al. Methods Mol Biol. 2017 1521: 153-166, which is incorporated by reference for this teaching. In some embodiments, the delivery vehicles is a polymer-lipid nanoparticle as described in Kaczmarek J C, et al. Angew Chem Int Ed Engl. 2016 55(44): 13808-13812, which is incorporated by reference for this teaching. In some embodiments, the delivery vehicle is a dendrimer-RNA nanoparticle as described in Chahal J S, et al. Proc Natd Acad Sci USA. 2016 1 13(29):E4133-42, which is incorporated by reference for this teaching. In some embodiments, the delivery vehicle is a poly(glycoamidoamine) brush as described in Dong Y, et al. Nano Lett. 2016 16(2):842-8, which is incorporated by reference for this teaching. In some embodiments, the delivery vehicle is a lipid-like nanoparticle as described in Eltoukhy A A, et al. Biomaterials. 2014 35(24):6454-61, which is incorporated by reference for this teaching. In some embodiments, the delivery vehicle is a low-molecular-weight polyamines and lipid nanoparticle as described in Dahlman J E, et al. Nat Nanotechnol. 2014 9(8):648-655, which is incorporated by reference for this teaching. In some embodiments, the delivery vehicle is a lipopeptide nanoparticle as described in Dong Y, et al. Proc Natl Acad Sci USA. 2014 111 (11):3955-60, which is incorporated by reference for this teaching. In some embodiments, the delivery vehicle is a lipid-modified aminoglycoside derivative as described in Zhang Y, et al. Adv Mater. 2013 25(33):4641-5, which is incorporated by reference for this teaching. In some embodiments, the delivery vehicle is a functional polyester as described in Yan Y, et al. Proc Natl Acad Sci USA. 2016 113(39):E5702-10, which is incorporated by reference for this teaching. In some embodiments, the delivery vehicle is a degradable dendrimers as described in Zhou K, et al. Proc Natl Acad Sci USA. 2016 113(3):520-5, which is incorporated by reference for this teaching. In some embodiments, the delivery vehicle is a lipocationic polyester as described in Hao J, et al. J Am Chem Soc. 2015 137(29):9206-9, which is incorporated by reference for this teaching. In some embodiments, the delivery vehicle is a nanoparticle with a cationic cores and variable shell as described in Siegwart D J, et al. Proc Natl Acad Sci USA. 2011 108(32): 12996-3001, which is incorporated by reference for this teaching. In some embodiments, the delivery vehicle is an amino-ester nanomaterial as described in Zhang X, et al. ACS Appl Mater Interfaces. 2017 9(30):25481-25487, which is incorporated by reference for this teaching. In some embodiments, the delivery vehicle is a polycationic cyclodextrin nanoparticle as described in Zuckerman J E, et al. Nucleic Acid Ther. 2015 25(2):53-64, which is incorporated by reference for this teaching. In some embodiments, the delivery vehicle is a cyclodextrin-containing polymer conjugate of camptothecin as described in Davis M E. Adv Drug Deliv Rev. 2009 61 (13): 1189-92, or Gaur S, et al. Nanomedicine. 2012 8(5):721-30, which are incorporated by reference for these teachings. In some embodiments, the delivery vehicle is an oligothioetheramide as described in Sorkin M R, et al. Bioconjug Chem. 2017 28(4):907-912, which is incorporated by reference for this teaching. In some embodiments, the delivery vehicle is a macrocycles as described in Porel M, et al. Nat Chem. 2016 June; 8(6):590-6, which is incorporated by reference for this teaching. In some embodiments, the delivery vehicle is a lipid nanoparticle as described in Alabi C A, et al. Proc Natl Acad Sci USA. 2013 110(32): 12881-6, which is incorporated by reference for this teaching. In some embodiments, the delivery vehicle is a poly(beta-amino ester) (PBAE) nanoparticle as described in Zamboni C G, et al. J Control Release. 2017 263: 18-28, which is incorporated by reference for this teaching. In some embodiments, the delivery vehicle is a poly(P-amino ester) (PBAE) as described in Green J J, et al. Acc Chem Res. 2008 41 (6):749-59, which is incorporated by reference for this teaching. In some embodiments, the delivery vehicle is a stable nucleic acid lipid particles (SNALP) as described in Semple S C, et al. Nat Biotechnol. 2010 28(2): 172-6, which is incorporated by reference for this teaching. In some embodiments, the material is an amino sugar. In one embodiment the material is GaINAc as described in Tanowitz M, et al. Nucleic Acids Res. 2017 Oct. 23; Nair J K, et al. Nucleic Acids Res. 2017 Sep. 15; and Zimmermann T S, et al. Mol Ther. 2017 Jan. 4:25(1):71-78, which are incorporated by reference for these teaching.

2. Conjugate Delivery Vehicles

In some embodiments, the delivery vehicle is a conjugate system. The core material can be a peptide, lipid, ssRNA, dsRNA, ssDNA, dsDNA, a polymer, a polymer/lipid combination, a peptide/lipid combination, or combinations thereof.

In one embodiment the reporter is ionically bonded to the conjugate delivery vehicle. The reporter can be bonded to the conjugate delivery system by hydrogen bonding, Watson-Crick base pairing, or hydrophobic interaction.

Exemplary reporters include, but are not limited to siRNA, nuclease protein, mRNA, nuclease mRNA, small molecules, and epigenetic modifiers. In one embodiment the reporter causes a detectable, phenotypic change in the cell. For example, the reporter can cause the cell to change morphology, metabolic activity, increase or decrease in gene expression, etc.

B. Formulating Delivery Vehicles

In one embodiment, the delivery vehicle used in the disclosed methods is a particulate delivery vehicle. For example the delivery vehicle can be nanoparticle including but not limited to a lipid nanoparticle. In one embodiment, the particulate delivery vehicle encapsulates the reporter and the chemical composition identifier. In other embodiments, the reporter, the chemical composition identifier, or both are conjugated to the delivery vehicle.

In one embodiment nanoparticles are formulated by combining a biomaterial with a synthetic or commercial lipid in a tube with an organic solvent such as 100% ethanol and mixing them. In a second tube, the reporter and the chemical composition identifier are combined and mixed, typically in a buffered solution. Next the content of the two tubes are mixed together to produce the nanoparticles. The biomaterial in tube one can be an ionizable lipid, a polymer, a peptide, nucleic acid, carbohydrate, etc. A variety of different formulations can be quickly produced using a microfluidic device as disclosed in Chen D, et al. (2012) Rapid discovery of potent siRNA-containing lipid nanoparticles enabled by controlled microfluidic formulation. J Am Chem Soc 134:6948-6951, which is incorporated by reference in its entirety.

In another embodiment, nucleic acids (mRNA, DNA barcodes, siRNA, and sgRNA) are diluted in a buffer, for example 10 mM citrate buffer, while lipid-amine compounds, alkyl-tailed PEG, cholesterol, and helper lipids were diluted in ethanol. For nanoparticle screens, the reporter and chemical composition identifier, for example DNA barcodes, are mixed at a 10:1 mass ratio. It will be appreciated that the mass ratio can be optimized for each run. Citrate and ethanol phases were combined in a microfluidic device by syringes (Hamilton Company) at a flow rate of 600 μL/min and 200 μL/min, respectively. All PEGs, cholesterol, and helper lipids were purchased from Avanti Lipids.

The biophysical and chemical characteristics of materials use to formulate the delivery vehicles. Parameters for optimization may include but are not limited to any of size, polymer composition, surface hydrophilicity, surface charge, and the presence, composition and density of targeting agents on the material surface. A library of delivery vehicles in which these or other parameters are varied may be produced using combinatorial techniques. Combinatorial techniques may also be used to provide a unique label for each material or population of materials. A large number of different formulations for the delivery vehicles can be achieved by varying lipid-amine compound, the molar amount of PEG, the structure of PEG, and the molar amount of cholesterol in the particles is varied among the particles.

1. Representative Polymers

The delivery vehicles can be formulated from a variety of materials. In some embodiments, the delivery vehicles contain helper lipids. Helper lipids contribute to the stability and delivery efficiency of the delivery vehicles. Helper lipids with cone-shape geometry favoring the formation hexagonal II phase can be used. An example is dioleoylphosphatidylethanolamine (DOPE) which can promote endosomal release of cargo. Cylindrical-shaped lipid phosphatidylcholine can be used to provide greater bilayer stability, which is important for in vivo application of LNPs. Cholesterol can be included as a helper that improves intracellular delivery as well as LNP stability in vivo. Inclusion of a PEGylating lipid can be used to enhance LNP colloidal stability in vitro and circulation time in vivo. In some embodiments, the PEGylation is reversible in that the PEG moiety is gradually released in blood circulation. pH-sensitive anionic helper lipids, such as fatty acids and cholesteryl hemisuccinate (CHEMS), can trigger low-pH-induced changes in LNP surface charge and destabilization that can facilitate endosomal release.

Representative materials that can be used to produce the disclosed delivery vehicles include, but are not limited to poly(ethylene glycol), cholesterol, 1,2-dioleoylsn-glycero-3-phosphoethanolamine (DOPE), 1-(1Z-hexadecenyl)-sn-glycero-3-phosphocholine, 1-O-1′-(Z)-octadecenyl-2-hydroxy-sn-glycero-3-phosphocholine, 1-(1Z-octadecenyl)-2-oleoyl-sn-glycero-3-phosphocholine, 1-(1Z-octadecenyl)-2-arachidonoyl-sn-glycero-3-phosphocholine, 1-O-1′-(Z)-octadecenyl-2-hydroxy-sn-glycero-3-phosphoethanolam ine, 1-(1Z-octadecenyl)-2-docosahexaenoyl-sn-glycero-3-phosphocholine, 1-(1Z-octadecenyl)-2-oleovl-sn-glycero-3-phosphoethanolamine, 1-(1Z-octadecenyl)-2-arachidonoyl-sn-glycero-3-phosphoethanolamine, 1-(1Z-octadecenyl)-2-docosahexaenoyl-sn-glycero-3-phosphoethanolamine, 1-palmitoyl-2-(5′-oxo-valeroyl)-sn-glycero-3-phosphocholine, 1-palmitoyl-2-(9′-oxo-nonanoyl)-sn-glycero-3-phosphocholine, 1-palmitoyl-2-glutaryl-sn-glycero-3-phosphocholine, 1-hexadecyl-2-azelaoyl-sn-glycero-3-phosphocholine, 1-palmitoyl-2-azelaoyl-sn-glycero-3-phosphocholine, 1-(10-pyrenedecanoyl)-2-glutaroyl-sn-glycero-3-phosphocholine, 1-(10-pyrenedecanoyl)-2-(5,5-dimethoxyvaleroyl)-sn-glycero-3-phosphocholine, 1-palmitoyl-2-glutaroyl-sn-glycero-3-phosphoethanolamine-N-[4-(dipyrrometheneboron difluoride)butanoyl] (ammonium salt), 1-palmitoyl-2-(5,5-dimethoxyvaleroyl)-sn-glycero-3-phosphoethanolamine-N-[4-(dipyrrometheneboron difluoride)butanoyl] (ammonium salt), 2-((2,3-bis(oleoyloxy)propyl)dimethylammonio)ethyl hydrogen phosphate, 2-((2,3-bis(oleoyloxy)propyl)dimettlylammonio)ethyl ethyl phosphate, i-oleoyl-2-cholestervlhemisuccinoyl-sn-glycero-3-phosphocholine, 1,2-dicholesterylhemisuccinoyl-sn-glycero-3-phosphocholine, 1-palmitoyl-2-cholesterylcarbonoyl-sn-glycero-3-phosphocholine, 1-palmitoyl-2-cholesterylhemisuccinoyl-sn-glycero-3-phosphocholine, 1-O-hexadecanyl-2-O-(9Z-octadecenyl)-sn-glycero-3-phosphocoline, 1-O-hexadecanyl-2-O-(9Z-octadecenyl)sn-glycero-3-phospho-(1′-rac-glycerol) (ammonium salt), 1-O-hexadecanyl-2-O-(9Z-octadecenyl)-sn-glycero-3-phosphoethanolarnine, 1-O-hexadecyl-sn-glycerol (HG), 1,2-di-O-phytanyl-sn-glycerol, 1,2-di-O-phytanyl-sn-glycero-3-phosphoethanolamine, 1,2-di-O-tetradecyl-sn-glycero-3-phospho-(1′-rac-glycerol), 1,2-di-O-hexyl-sn-glycero-3-phosphocholine, 1,2-di-O-dodecyl-sn-glycero-3-ptIosphocholine, 1,2-di-O-tridecyl-sn-glycero-3-phosphocholine, 1,2-di-O-hexadecyl-sn-glycero-3-phosphocholine, 1,2-di-O-octadecyl-sn-glycero-3-phosphocholine, 1,2-di-O-(9Z-octadecenyl)-sn-glycero-3-phosphocholine, 1,2-di-O-phytanyl-sn-glycero-3-phosphocholine, 1-O-octadecyl-2-O-methyl-sn-glycero-3-phosphocholine, 1′,3′-bis[1,2-dimyristoyl-sn-glycero-3-ptlospho]-sn-glycerol, 1′,3′-bis[1,2-dimyristoleoyl-sn-glycero-3-phospho]-sn-glycerol, 1′,3′-bis[1,2-dipalmitoleoyl-sn-glycero-3-phospho]-sn-glycerol, 1′,3′-bis[1,2-distearoyl-sn-glycero-3-ptlospho]-sn-glycerol, 1′,3′-bis[1,2-dioleoyl-sn-glycero-3-phospho]-sn-glycerol, 1,3′-bis[1,2-dipalmitoyl-sn-glycero-3-phospho]-sn-glycerol, 1′,3′-bis[1-palmitoyl-2-oleoyl-sn-glycero-3-phospho]-sn-glycerol, 1-palmitoyl-2-oleoyl-sn-glycero-3-phospho-(1′-myo-inositol-4′-phosphate), 1-stearoyl-2-arachidonoyl-sn-glycero-3-phospho-(1′-myo-inositol-4′-phosphate), 1,2-dioctanoyl-sn-glycero-3-(phosphoinositol-3-phosphate), 1,2-dioctanoyl-sn-glycero-3-phospho-(1′-myo-inositol-3′,4′,5′-trisphosphate), 1,2-dioctanoyl-sn-glycero-3-phospho-(1′-myo-inositol-4′,5′-bisphosphate), 1,2-dioctanoyl-sn-glycero-3-phospho-(1′-myo-inositol-3′,4′bisphosphate), 1,2-dioctanoyl-sn-glycero-3-phospho-(1′-myo-inositol-4′-phosphate), 1,2-dioctanoyl-sn-glycero-3-phospho-(1′-myo-inositol), 1,2-dihexanoyl-sn-glycero-3-phospho-(1′-myo-inositol-3′,4′,5′-trisphosphate), 1,2-dihexanoyl-sn-glycero-3-phospho-(1′-myo-inositol-3′,5′-bisphosphate), 1-stearoyl-2-arachidonoyl-sn-glycero-3-phospho-(1′-myo-inositol-3′,4′,5′-trisphosphate), 1-stearoyl-2-arachidonoyl-sn-glycero-3-phospho-(1′-myo-inositol-4′,5′-bisphosphate), 1-stearoyl-2-arachidonoyl-sn-glycero-3-phospho-(1′-myo-inositol-3′,5′-bisphosphate), 1,2-dioleoyl-sn-glycero-3-phospho-(1′-myo-inositol-3′,4′,5′-trisphosphate), 1,2-dioleoyl-sn-glycero-3-phospho-(1′-myo-inositol-4′,5′-bisphosphate), 1,2-dioleoyl-sn-glycero-3-phospho-(1′-myoinositol-3′,5′-bisphosphate), 1,2-dioleoyl-sn-glycero-3-phospho-(1′-myo-inositol-3′,4′bisphosphate), 1,2-dioleoyl-sn-glycero-3-phospho-(1′-myo-inositol-5′-phosphate), 1,2-dioleoyl-sn-glycero-3-phospho-(1′-myo-inositol-4′-phosphate), 1,2-dioleoyl-sn-glycero-3-phospho-(1′-myo-inositol-3′-phosphate), 1,2-dioleoyl-sn-glycero-3-phospho-(1′-myo-inositol), 1-stearoyl-2-arach idonoyl-sn-glycero-3-phosphoinositol, 1,2-d istearoyl-sn-glycero-3-phosphoinositol, 1-palmitoyl-2-oleoyl-sn-glycero-3-phosphoinositol, 1,2-dipalmitoyl-sn-glycero-3-phospho-(1′-myo-inositol), 1-oleoyl-2-(6-((4,4-difluoro-1,3-dimethyl-5-(4-methoxyphenyl)-4-bora-3a,4a-diaza-s-indacene-2-propionyl)amino)hexanoyl)-sn-glycero-3-phosphoinositol-4,5-bisphosphate, 1-oleoyl-2-hydroxy-sn-glycero-3-phospho-(1′-myo-inositol), 1-tridecanoyl-2-hydroxy-snglycero-3-phospho-(1′-myo-inositol), 1-palmitoyl-2-hydroxy-sn-glycero-3-phosphoinositol, 1-(10Z-heptadecenoyl)-2-hydroxy-sn-glycero-3-phospho-(1′-myoinositol), 1-stearoyl-2-hvdrm,y-sn-glycero-3-phospl10inositol, 1-arachidonoyl-2-hydroxy-sn-glycero-3-phosphoinositol, D-myo-inositol-1,3,4-trisphosphate, D-myoinositol-1,3,5-triphosphate, D-myo-inositol-1,4,5-triphosphate, D-myo-inositol-1,3,4,5-tetraphosphate, 1-(10Z-heptadecenoyl)-2-hydroxy-sn-glycero-3-[phospho-L-serine], or any combination thereof.

2. Biocompatible Polymers

In certain embodiments, the delivery vehicles are fabricated from or contain biocompatible polymers. A variety of biodegradable and/or biocompatible polymers are well known to those skilled in the art. Exemplary synthetic polymers suitable for use with the disclosed compositions and methods include but are not limited to poly(lactide), poly(glycolide), poly(lactic co-glycolic acid), poly(arylates), poly(anhydrides), poly(hydroxy acids), polyesters, poly(ortho esters), polycarbonates, poly(propylene fumerates), poly(caprolactones), polyamides, polyphosphazenes, polyamino acids, polyethers, polyacetals, polylactides, polyhydroxyalkanoates, polyglycolides, polyketals, polyesteramides, poly(dioxanones), polyhydroxybutyrates, polyhydroxyvalyrates, polycarbonates, polyorthocarbonates, polyvinyl pyrrolidone), biodegradable polycyanoacrylates, polyalkylene oxalates, polyalkylene succinates, poly(malic acid), poly(methyl vinyl ether), poly(ethylene imine), poly(acrylic acid), poly(maleic anhydride), biodegradable polyurethanes and polysaccharides. In certain embodiments, the materials include polyethylene glycol (PEG). In certain embodiments, the polymer used to make the materials is PEGylated (i.e., conjugated to a polyethylene glycol moiety).

In some embodiments, the delivery vehicle is formed from material identified as Generally Recognized as Safe (GRAS) by the FDA.

3. Naturally-Occurring Polymers

Naturally-occurring polymers, such as polysaccharides and proteins, may also be employed to produce the disclosed delivery vehicles. Exemplary polysaccharides include alginate, starches, dextrans, celluloses, chitin, chitosan, hyaluronic acid and its derivatives; exemplary proteins include collagen, albumin, and gelatin. Polysaccharides such as starches, dextrans, and celluloses may be unmodified or may be modified physically or chemically to affect one or more of their properties such as their characteristics in the hydrated state, their solubility, or their half-life in vivo. In certain embodiments, the materials do not include protein.

In other embodiments, the polymer includes polyhydroxy acids such as polylactic acid (PLA), polyglycolic acid (PGA), their copolymers poly(lactic-co-glycolic acid) (PLGA), and mixtures of any of these. In certain embodiments, the materials include poly(lactic-co-glycolic acid) (PLGA). In certain embodiments, the materials include poly(lactic acid). In certain other embodiments, the materials include poly(glycolic acid). These polymers are among the synthetic polymers approved for human clinical use as surgical suture materials and in controlled release devices. They are degraded by hydrolysis to products that can be metabolized and excreted. Furthermore, copolymerization of PLA and PGA offers the advantage of a large spectrum of degradation rates from a few days to several years by simply varying the copolymer ratio of glycolic acid to lactic acid, which is more hydrophobic and less crystalline than PGA and degrades at a slower rate.

Non-biodegradable polymers may also be used to produce materials. Exemplary non-biodegradable, yet biocompatible polymers include polystyrene, polyesters, non-biodegradable polyurethanes, polyureas, polyvinyl alcohol), polyamides, poly(tetrafluoroethylene), poly(ethylene vinyl acetate), polypropylene, polyacrylate, non-biodegradable polycyanoacrylates, non-biodegradable polyurethanes, polymethacrylate, poly(methyl methacrylate), polyethylene, polypyrrole, polyanilines, polythiophene, and poly(ethylene oxide).

4. Functionalized Polymers

Any of the above polymers may be functionalized with a poly(alkylene glycol), for example, poly(ethylene glycol) (PEG) or poly(propyleneglycol) (PPG), or any other hydrophilic polymer system. Alternatively or in addition, they may have a particular terminal functional group, e.g., poly(lactic acid) modified to have a terminal carboxyl group so that a poly(alkylene glycol) or other material may be attached. Exemplary PEG-functionalized polymers include but are not limited to PEG-functionalized poly(lactic acid), PEG-functionalized poly(lactic-co-glycolic acid), PEG-functionalized poly(caprolactone), PEG-functionalized poly(ortho esters), PEG-functionalized polylysine, and PEG-functionalized poly(ethylene imine). When used in formulations for oral delivery, poly(alkylene glycols) are known to increase the bioavailability of many pharmacologically useful compounds, partly by increasing the gastrointestinal stability of derivatized compounds. For parenterally administered pharmacologically useful compounds, including particle delivery systems, poly(alkylene glycols) are known to increase stability, partly by decreasing optimization of these compounds, thereby reducing immunogenic clearance, and partly by decreasing non-specific clearance of these compounds by immune cells whose function is to remove foreign material from the body. Poly(alkylene glycols) are chains may be as short as a few hundred Daltons or have a molecular weight of several thousand or more.

Co-polymers, mixtures, and adducts of any of the above modified and unmodified polymers may also be employed. For example, amphiphilic block co-polymers having hydrophobic regions and anionic or otherwise hydrophilic regions may be employed. Block co-polymers having regions that engage in different types of non-covalent or covalent interactions may also be employed. Alternatively or in addition, polymers may be chemically modified to have particular functional groups. For example, polymers may be functionalized with hydroxyl, amine, carboxy, maleimide, thiol, N-hydroxy-succinimide (NHS) esters, or azide groups. These groups may be used to render the polymer hydrophilic or to achieve particular interactions with materials that are used to modify the surface as described below.

One skilled in the art will recognize that the molecular weight and the degree of cross-linking may be adjusted to control the decomposition rate of the polymer. Methods of controlling molecular weight and cross-linking to adjust release rates are well known to those skilled in the art.

5. Non-Polymer Materials

Delivery vehicles may also be produced from non-polymer materials, e.g., metals, and semiconductors. For example, where it is desired to provide a contrast or imaging agent to a particular tissue, it may not be necessary to combine a particulate agent with a polymer carrier.

The surface chemistry of the delivery vehicles may be varied using any technique known to the skilled artisan. Both the surface hydrophilicity and the surface charge may be modified. Some methods for modifying the surface chemistry of polymer materials are discussed above. Silane or thiol molecules may be employed to tether particular functional groups to the surface of polymer or non-polymer materials. For example, hydrophilic (e.g., thiol, hydroxyl, or amine) or hydrophobic (e.g., perfluoro, alkyl, cycloalkyl, aryl, cycloaryl) groups may be tethered to the surface. Acidic or basic groups may be tethered to the surface of the materials to modify their surface charge. Exemplary acidic groups include carboxylic acids, nitrogen-based acids, phosphorus based acids, and sulfur based acids. Exemplary basic groups include amines and other nitrogen containing groups. The pKa of these groups may be controlled by adjusting the environment of the acidic or basic group, for example, by including electron donating or electron withdrawing groups adjacent to the acidic or basic group, or by including the acidic or basic group in a conjugated or non-conjugated ring. Alternatively, materials may be oxidized, for example, using peroxides, permanganates, oxidizing acids, plasma etching, or other oxidizing agents, to increase the density of hydroxyl and other oxygenated groups at their surfaces. Alternatively or in addition, borohydrides, thiosulfates, or other reducing agents may be used to decrease the hydrophilicity of the surface.

6. Size Range

The delivery vehicles may be any size that permits cells to uptake the particles. For example, the particles can have a diameter of about 1 nm to about 1000 μm, or about 1 and about 50 nm, or 50 to 100 nm, or about 100 to about 500 nm, or about 500 to about 1000 nm, or about 1 μm to about 10 μm.

In some embodiments, the screening method is used to screen microparticles (having a diameter between 1 and 10 microns) or nanoparticles (having a diameter between 1 and 1000 nm) for characteristics suitable for delivering a functional bioactive agent to a cell, tissue, or organ of interest.

The number of delivery vehicles characterized per run of the assay can be at least 50, 100, 150, 200, 250, 300, 350, 400, 450, 500 or more depending on the size of the non-human mammal used in the assay.

7. Targeting Agents

In some embodiments, targeting agents may be employed to more precisely direct the delivery vehicles to a tissue or cell of interest. Therefore, the disclosed delivery vehicles can contain a tissue-targeting moiety, a cell-targeting moiety, a receptor-targeting moiety, or any combination thereof

One skilled in the art will recognize that the tissue of interest need not be healthy tissue but may be a tumor or particular form of damaged or diseased tissue, such as areas of arteriosclerosis or unstable antheroma plaque in the vasculature. Targeting agents may target any part or component of a tissue. For example, targeting agents may exhibit an affinity for an epitope or antigen on a tumor or other tissue cell, an integrin or other cell-attachment agent, an enzyme receptor, an extracellular matrix material, or a peptide sequence in a particular tissue. Targeting agents may include but are not limited to antibodies and antibody fragments (e.g. the Fab, Fab′, or F(ab′)2 fragments, or single chain antibodies), nucleic acid ligands (e.g., aptamers), oligonucleotides, oligopeptides, polysaccharides, low-density lipoproteins (LDLs), folate, transferrin, asialycoproteins, carbohydrates, polysaccharides, sialic acid, glycoprotein, or lipid. Targeting agents may include any small molecule, bioactive agent, or biomolecule, natural or synthetic, which binds specifically to a cell surface receptor, protein or glycoprotein found at the surface of cells. In some embodiments, the targeting agent is an oligonucleotide sequence. In certain embodiments, the targeting agent is an aptamer. In some embodiments, the targeting agent is a naturally occurring carbohydrate molecule or one selected from a library of carbohydrates. Libraries of peptides, carbohydrates, or polynucleotides for use as potential targeting agents may be synthesized using techniques known to those skilled in the art. Various macromolecule libraries may also be purchased from companies such as Invitrogen and Cambridge Peptide.

The targeting agent may be conjugated to the material by covalent interactions. For example, a polymeric material may be modified with a carboxylate group, following which an aminated targeting agent, or one that is modified to be aminated, is coupled to the polymer using a coupling reagent such as EDC or DCC. Alternatively, polymers may be modified to have an activated NHS ester which can then be reacted with an amine group on the targeting agent. Other reactive groups that may be employed to couple targeting agents to materials include but are not limited to hydroxyl, amine, carboxyl, maleimide, thiol, NHS ester, azide, and alkyne. Standard coupling reactions may then be used to couple the modified material to a second material having a complementary group (e.g., a carboxyl modified targeting agent coupled to an aminated polymer). Materials fabricated from inorganic materials may be modified to carry any of these groups using self-assembled monolayer forming materials to tether the desired functional group to the surface.

Alternatively, the targeting agents can be attached to the materials directly or indirectly via non-covalent interactions. Non-covalent interactions include but are not limited to electrostatic Interactions, affinity Interactions, metal coordination, physical adsorption, host-guest interactions, and hydrogen bonding interactions.

8. Nucleic Acid Bar Codes

One embodiment provides a nucleic acid bar code. The nucleic acid barcodes can be rationally designed to increase DNA polymerase access and so that DNA secondary structure on the forward and reverse primer sites are minimized and G-quadruplex formation is minimized by separating the fully randomized nucleotide region.

One embodiment provides a nucleic acid barcode according to the following formula

R1-R2-R3-R4-R5-R6-R7-R8-R1

wherein R1 represents 1, 2, 3, 4, 5, 6, 7, 8, 9, or 10 nucleotides with phosphorothioate linkages,
R2 represents a forward universal primer binding site,
R3 represents a spacer,
R4 represents a digital droplet PCR probe binding site,
R5 represents a random nucleotide sequence;
R6 represents a nucleic acid barcode sequence;
R7 represents a random nucleic acid sequence;
R8 represents a reverse universal primer binding site.

In one embodiment, the nucleic acid barcode does not contain phosphorothioate linkages.

In another embodiment, R3 has the following sequence NHNW, wherein N is A, T, G, or C; W is A or T; and H is A, T, or C. In one embodiment R5 has the following sequence NWNH and R7 has the following sequence NWH, wherein N is A, T, G, or C; W is A or T; and H is A, T, or C.

As used herein, the term “nucleic acid barcode” refers to an oligonucleotide having a nucleic acid sequence that contains a series of nucleotides (“barcode sequence”) unique to the barcode and optionally a series of nucleotides common to other barcodes. The common nucleotides can be used, for example, to isolate and sequence the barcode. Therefore, in some cases, the barcode sequence is flanked by upstream and downstream primer sites, such as, for example, universal primer sites. The polynucleotide can include a DNA nucleotide, an RNA nucleotide, or a combination thereof. Each delivery vehicle formulation is paired with its own unique nucleic acid barcode. The unique nucleic acid barcode is paired to the chemical composition of the delivery vehicle formulation and by sequencing the nucleic acid barcode, one can identify the specific chemical composition used to produce that specific vehicle delivery formulation.

The barcode can contain 5 to 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 45 nucleotides in length, about 5 to about 40 nucleotides. The nucleic acid barcodes can be covalently or non-covalently attached to the disclosed delivery vehicle. In some embodiments, the nucleic acid barcode is encapsulated by the delivery vehicle.

Another embodiment provides a pharmaceutical composition containing one or more of the nucleic acid barcodes described herein.

A number of embodiments of the invention have been described.

Nevertheless, it will be understood that various modifications may be made without departing from the spirit and scope of the invention. Accordingly, other embodiments are within the scope of the following claims.

### EXAMPLES

### Example 1: Modifying a Commonly Expressed Endocytotic Receptor Retargets Nanoparticles In Vivo

### Materials and Methods

The following example makes use of barcoding and screening techniques that have been described in the '561 Application.

### LNP Formulation

The lipid nanoparticle components were dissolved in 100% ethanol at specified lipid component molar ratios. The nucleic acid (NA) cargo was dissolved in 10 mM citrate, 100 mM NaCl, pH 4.0, resulting in a concentration of NA cargo of approximately 0.22 mg/mL. In some embodiments, NA cargos consist of both a functional NA (e.g. siRNA, anti-sense, expressing DNA, mRNA) as well as a reporter DNA barcode (as previously described Sago, 2018 PNAS) mixed at mass ratios of 1:10 to 10:1 functional NA to barcode. In this experiment, the functional nucleic acid was siRNA targeted to the gene CD45, and was mixed at a mass ratio of 10:1. This siRNA sequence is cross-reactive between mouse, rat, NHP, and human. The LNPs were formulated with a total lipid to NA mass ratio of 11.7. The LNPs were formed by microfluidic mixing of the lipid and NA solutions using a Precision Nanosystems NanoAssemblr Spark and Benchtop instruments, according to the manufacturers protocol. A 2:1 or 3:1 ratio of aqueous to organic solvent was maintained during mixing using differential flow rates. After mixing, the LNPs were collected, diluted in PBS (approximately 1:1 v/v), and further buffer exchange was conducted using dialysis in PBS at 4° C. for 8 to 24 hours against a 20 kDa filter. After this initial dialysis, each individual LNP formulation was characterized via DLS to measure the size and polvdispersity, and the pKa of a subpopulation of LNPs were measured via TNS assay. LNPs falling within specific diameter and polydispersity ranges were pooled, and further dialyzed against PBS at 4° C. for 1 to 4 hours against a 100 kDa dialysis cassette. After the second dialysis, LNPs were sterile filtered using 0.22 μM filter and stored at 4° C. for further use.

### LNP Characterization

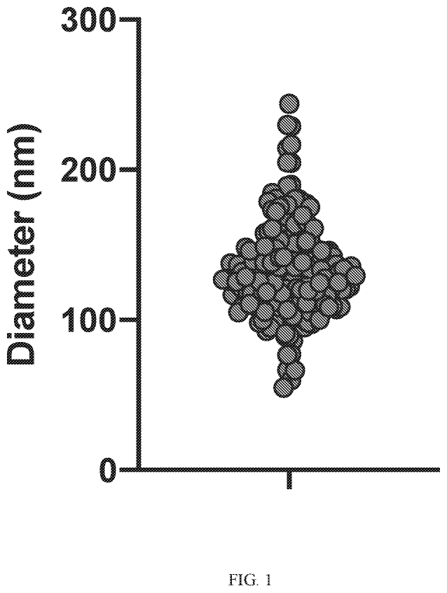

DLS—LNP hydrodynamic diameter and polydispersity percent (PDI %) were measured using high throughput dynamic light scattering (DLS) (DynaPro plate reader II, Wyatt). LNPs were diluted 1×PBS to an appropriate concentration and analyzed. FIG. 1 shows the diameter distribution of 192 LNPs formulated to carry siCD45 and DNA barcodes at a mass ratio of 10:1, each dot is the diameter of a distinct LNP.

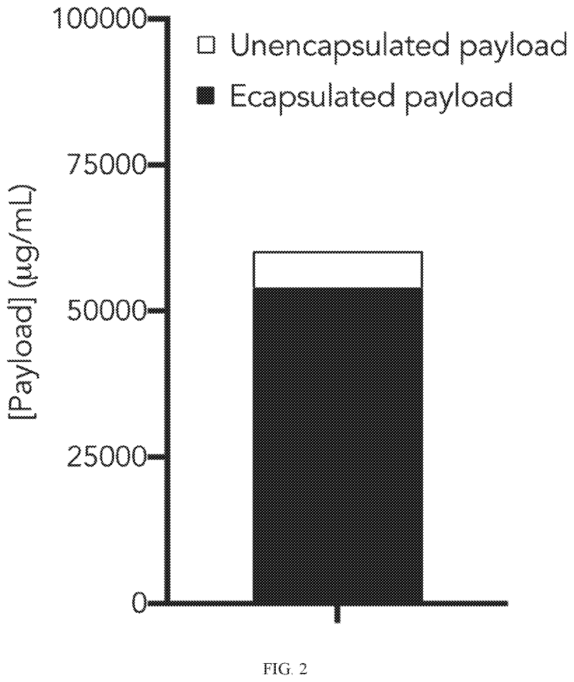

Concentration & Encapsulation Efficiency—Concentration of NA was determined by Qubit microRNA kit (for siRNA) or HS RNA kit (for mRNA) per manufacturer's instructions. Encapsulation efficiency was determined by measuring unlysed and lysed LNPs. FIG. 2 shows the concentration of encapsulated and unencapsulated siRNA of the pool of 192 LNPs.

### LNP Dosing for Rats

LNPs were dosed into male Sprague Dawley rats at a dose of 1.5 mg/kg siRNA payload by infusion into the tail-vein. As noted above, in other embodiments LNPs can be administered by bolus injection into the tail vein or by other routes of administration including, subcutaneous, intramuscular, intradermal, intrathecal, intravitreal, subretinal, intranasal, or nebulization.

### FACS for Rats

Select tissues (e.g. liver, lung, heart) were mechanically and enzymatically digested using a mixture of proteinases, then passed through a 70 uM filter to generate single cell suspensions. Other tissues (e.g. spleen) were mechanically digested to generate single cell suspensions. All tissues were treated with ACK buffer to lyse red blood cells, and then stained with fluorescently-labeled antibodies for flow cytometry and FACS sorting. All antibodies were commercially available antibodies. Using a BD FACSMelody (Becton Dickinson), all samples were acquired via flow cytometry to generate gates prior to sorting.

In general, the gating structure was size→singlet cells→live cells→cells of interest. T cells were defined as CD3+, monocytes were defined as CD11b+, and B cells were defined as CD19+. In the liver, LSECs were defined as CD31+, Kupffer cells as CD11 b+ and hepatocytes as CD31−/CD45−. For siRNA studies, we gated for downregulation of the target gene (CD45). Tissues from vehicle (saline)-dosed rats were used to set the gates for sorting. Up to 20,000 cells of each cell subset with the correct phenotype was sorted into 1×PBS. After sorting, cells were pelleted via centrifugation and DNA was extracted using Quick Extract DNA Extraction Solution (Lucigen) according to manufacturers protocol. DNA was stored at −20° C. until sequencing.

### DNA Sequencing

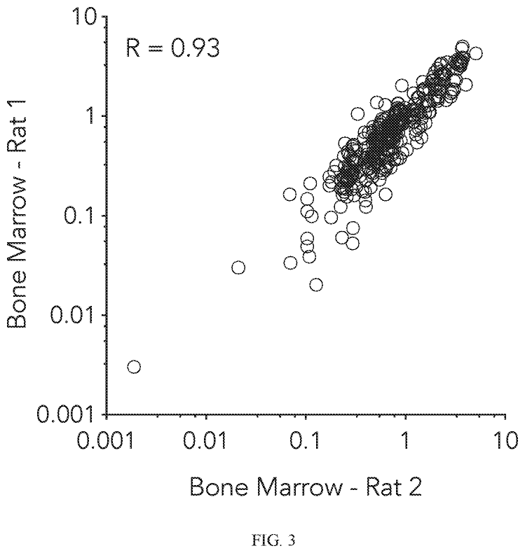

DNA (genomic and DNA barcodes) were isolated using QuickExtract (Lucigen) and sequenced using Illumina MiniSeq as previously described (Sago et al. PNAS 2018, Sago et al. JACS 2018, Sago, Lokugamage et al. Nano Letters 2018), normalizing frequency DNA barcode counts in FACS isolated samples to frequency in injected input. These data are plotted as ‘Normalized Fold Above Input’ wherein the value ‘1’ represents a LNP appearing at the same frequency in the FACS isolated sample as it did in the injection volume, representing that it displayed neutral tropism to the cell-type measured relative to other LNP populations in that same injection pool. FIG. 3 shows the normalized fold above input relating the delivery of each of the 201 chemically distinct LNPs in bone marrow tissue extracted from two distinct rats that had been administered a pool of 201 LNPs carrying siRNA targeting CD45 and DNA barcodes at a siRNA dose of 1.5 mg/kg. In FIG. 3, each point represents a distinct LNP.

### Screening in Rats and Non-Human Primates

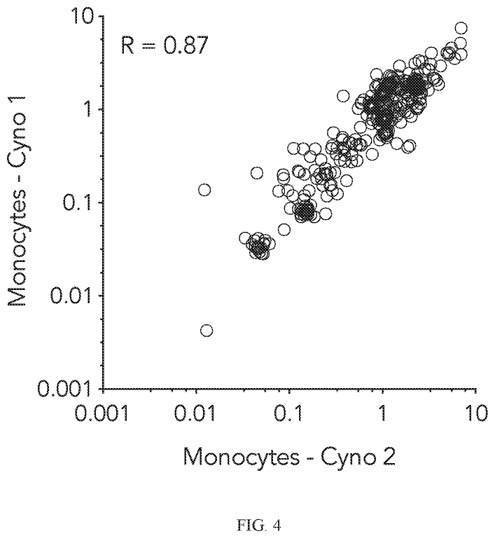

For the screening of LNPs in NHPs, the same general procedure of LNP formulation, LNP characterization, dosing, cell-type isolation, and DNA sequencing was conducted. As NHPs can be significantly larger than rats, the total mass of siRNA may be scaled up according to animal mass or surface area. FIG. 4 shows the normalized fold above input relating the delivery of each of the 201 chemically distinct LNPs in bone marrow monocytes FACS from two distinct NHPs that had been administered a pool of 201 LNPs carrying siRNA targeting CD45 and DNA barcodes at a siRNA dose of 1.5 mg/kg. In FIG. 4, each point represents a distinct LNP.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method of characterizing delivery vehicles for delivery of an agent, comprising:
(a) formulating multiple lipid nanoparticle (LNP) delivery vehicles having different chemical compositions, wherein each different LNP delivery vehicle comprises:
(i) a biologically active molecule that generates a detectable signal when delivered by the LNP delivery vehicle to the cytoplasm of cells of at least two species of non-human mammals; and
(ii) a chemical composition identifier that identifies the chemical compositions of each of the LNP delivery vehicles;
(b) administering multiple LNP delivery vehicles to multiple tissues of at least one of the species of non-human mammal;
(c) sorting cells from the multiple tissues of the non-human mammal that generate the detectable signal from cells that do not generate the detectable signal, wherein the cells that generate the detectable signal are also sorted based on the presence or absence of a cell surface protein that is indicative of tissue type or cell type; and
(d) identifying the chemical composition identifier in the sorted cells that generate the detectable signal to determine the chemical composition of the LNP delivery vehicle in the sorted cells to correlate the chemical composition of the LNP delivery vehicles with the tissue or cell type containing the LNP delivery vehicles.

2. The method of claim 1, wherein the LNP delivery vehicles further comprise the agent to be delivered.

3. The method of claim 1 or 2, wherein the two species of non-human mammals are selected from mouse, rat and non-human primate.

4. The method of any one of claims 1-3, wherein the agent is a nucleic acid agent.

5. The method of claim 4, wherein the nucleic acid agent comprises RNA, DNA, or a combination of RNA and DNA.

6. The method of any one of claims 1-5, wherein the detectable signal is indicative of down regulation of a gene typically expressed in the cells.

7. The method of claim 6, wherein the down regulation results in reduced expression of one or more of: beta-2-microglobulin, CD47, CD81, AP2S1, LGALS9, ITGB1, ITGA5, CD45, TIE2, MGAT4B, MGAT2, VAMP3, and GPAA1.

8. The method of claim 6, wherein the biologically active molecule that generates a detectable signal is siRNA, an antisense oligonucleotide or a DNA transgene.

9. The method of claim 8, wherein the DNA transgene expresses a shRNA.

10. The method of any one of claims 1-5, wherein the detectable signal is indicative of up regulation of a gene typically expressed in the cells.

11. The method of claim 9, wherein the biologically active molecule that generates a detectable signal is mRNA or a DNA transgene.

12. The method of claim 11, wherein the mRNA is a modified mRNA that enhances generation of the detectable signal and/or decreases immunogenicity as compared to unmodified mRNA.

13. The method of claim 12, wherein the modified mRNA comprises one or more of a 5′-endcap, a 5′-untranslated region (UTR), a 3′-UTR, 3′-polyadenylation, codon optimization and base modifications.

14. The method of any one of claims 1-13, wherein the chemical composition identifier is a nucleic acid barcode.

15. The method of claim 14, further comprising sequencing the nucleic acid barcodes to identify the chemical compositions of the LNP delivery vehicles.

16. The method of any one of claims 1-15, wherein the non-human mammal to which the multiple LNP delivery vehicles are administered is a non-human primate.

17. The method of any one of claims 1-15, wherein the biologically active molecule that generates a detectable signal comprises a DNA transgene.

18. The method of claim 17, wherein the biologically active molecule that generates a detectable signal further comprises at least one selected from a cell-specific promoter, a small RNA promoter, a 3′-UTR, 3′-polyadenylation, a small RNA polymerase terminal, and the chemical composition identifier.

19. The method of claim 18, wherein the chemical composition identifier comprises a nucleic acid barcode and, optionally, a barcode tag.

20. The method of claim 17, wherein the biologically active molecule that generates a detectable signal is represented by at least one selected from:
5′-Promoter-Transgene-3′UTR & PolyA-Small RNA pol term-Tag for BC-Barcode-Small RNA Promoter-3′;
5′-Small RNA poly term-Tag for BC-Barcode-Small RNA Promoter-Promoter-Transgene-3′UTR & PolyA-3′;
5′-Small RNA Promoter-Barcode-Tag for BC-Small RNA pol term-Promoter-Transgene-3′UTR & PolyA-3′;
5′-Barcode-Promoter-Transgene-3′UTR & PolyA-3′; and
5′-Promoter-Transgene-3′UTR & PolyA-3′+Barcode.
